(19) United States
(12) Patent Application Publication
(10) Pub. No.: US 2008/0175892 A1
(43) Pub. Date: Jul. 24, 2008

(54) Nanostructure-Drug Conjugates

(76) Inventors: Lon J. Wilson, Houston, TX (US); Michael G. Rosenblum, Sugar Land, TX (US)

Correspondence Address: BAKER BOTTS, LLP, 910 LOUISIANA, HOUSTON, TX, 77002-4995, US

(21) Appl. No.: 11/841,524

(22) Filed: Aug. 20, 2007

Related U.S. Application Data
(60) Provisional application No. 60/822,837, filed on Aug. 18, 2006.

(51) Int. Cl. A61K 9/127; A61K 47/42; A61K 9/12; A61P 35/00; A61K 31/337

(52) U.S. Cl. 424/450; 514/773; 514/449; 424/45; 977/734; 977/750

(57) ABSTRACT

Nanostructure-drug conjugates and associated methods of use are provided. In one embodiment, a nanostructure-drug conjugate comprising a Cₙ, a crosslinker, and a drug is provided, wherein Cₙ refers to a fullerene moiety or nanotube comprising n carbon atoms. A method of treating cancer is also provided, comprising administering a therapeutically effective amount of a nanostructure-drug conjugate comprising: a Cₙ, a crosslinker, and a drug, wherein Cₙ refers to a fullerene moiety comprising n carbon atoms to a mammal.

### CROSS-REFERENCE TO RELATED APPLICATIONS

This application claims the benefit of U.S. Provisional Application No. 60/822,837, filed Aug. 18, 2006, incorporated herein by reference.

### STATEMENT OF GOVERNMENT INTEREST

This disclosure was developed at least in part with support under grant number CA093941 awarded by the National Institutes of Health. The government has certain rights in the invention.

### BACKGROUND

Liposome aerosol delivery has been successfully used for lung cancer therapy employing a variety of lipophilic chemotherapeutics. Small particle liposome aerosol treatment consists of lipid-soluble or water-soluble anti-cancer drugs incorporated into liposomes, which are administered from aqueous dispersions in a jet nebulizer. Aerosols, generated upon nebulization, enable targeted delivery onto surfaces of the respiratory tract. The deposited liposomes subsequently release drug locally within the lung or into the blood circulation with delivery to extra-pulmonary tissue. If the drug is lipid soluble, it will associate with the lipid molecules in a manner specific to the lipid employed, the anti-cancer drug employed and possibly it may be modified further by various soluble constituents which may be included in the suspending aqueous medium. Such soluble constituents may include buffering salts and possibly inositol to enhance the synthesis and secretion of surfactant phospholipid in lung tissue and to minimize respiratory distress already present or that which might result from the aerosol treatment. However, these drugs exhibit rapid clearance from the lungs after cessation of aerosol delivery, which reduces therapeutic efficiency of the drug.

Fullerene (C₆₀) and carbon nanotube materials have been studied extensively for use in nanomedicine and show great promise. Water-soluble C₆₀ derivatives are now commonplace and the discovery that water-soluble C₆₀ derivatives can cross cell membranes and even produce transfection has accelerated interest in utilizing C₆₀ for therapeutic medicine. Further, several water-soluble C₆₀ derivatives have demonstrated acceptable cytotoxicity for drug-delivery applications.

### DRAWINGS

Some specific example embodiments of the disclosure may be understood by referring, in part, to the following description and the accompanying drawings.

FIG. 1 shows structures of Paclitaxel (1) and paclitxel-2′-succinate (2)

FIG. 2 shows synthesis of Paclitaxel-Fullerene Conjugate.

FIG. 3 shows kinetics of hydrolysis of paclitaxel-fullerene conjugate in bovine plasma at 37° C. Conjugate was incubated in the presence of bovine plasma, and the concentrations of conjugate and paclitaxel were determined at the indicated timepoints using reverse phase HPLC. The curved fits are only to guide the eyes.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to compositions and methods related to carbon nanostructures. More particularly, the present disclosure relates to nanostructure-drug conjugates and associated methods of use.

In one embodiment, the present disclosure relates to a nanostructure-drug conjugate comprising: a Cₙ, a crosslinker, and a drug, wherein Cₙ refers to a fullerene moiety or nanotube comprising n carbon atoms. As used herein, the term “drug” refers to refers to any chemical compound that is used for one or more of the prevention, diagnosis, treatment, and cure of disease, for the relief of pain, or to control or improve any physiological or pathological disorder in humans or animals. As used herein, the term “crosslinker” refers to anything that is capable of forming links between molecular chains to form a connected molecule.

The nanostructure-drug conjugate may further comprise a targeting agent. As used herein, the term “targeting agent” refers to a moiety comprising an antigen-binding site and that is linked to the Cₙ. As used herein, the term “antigen” refers to a chemical compound or a portion of a chemical compound which can be recognized by a specific chemical reaction or a specific physical reaction with another molecule. The antigen-recognition site of an antibody is an exemplary, but non-limiting, antigen-binding site.

Cₙ refers to a fullerene moiety comprising n carbon atoms or a nanotube moiety comprising at least n carbon atoms. Examples of suitable Cₙ compounds for use in conjunction with the compositions of the present disclosure include, but are not limited to, buckminsterfullerenes, gadofullerenes, single walled carbon nanotubes (SWNTs), and ultra-short carbon nanotubes (US-tubes). Buckminsterfullerenes, also known as fullerenes or more colloquially, buckyballs, are closed-cage molecules consisting essentially of sp²-hybridized carbons. Fullerenes are the third form of pure carbon, in addition to diamond and graphite. Typically, fullerenes are arranged in hexagons, pentagons, or both. Most known fullerenes have 12 pentagons and varying numbers of hexagons, depending on the size of the molecule. Common fullerenes include C₆₀ and C₇₀ (e.g. n=60 or n=70), although fullerenes comprising up to about 400 carbon atoms are also known.

SWNTs, also known as single walled tubular fullerenes, are cylindrical molecules consisting essentially of SP² hybridized carbons. In defining the size and conformation of single-walled carbon nanotubes, the system of nomenclature described by Dresselhaus et al., Science of Fullerenes and Carbon Nanotubes, Ch. 19. will be used. Single walled tubular fullerenes are distinguished from each other by a double index (x,y), where x and y are integers that describe how to cut a single strip of hexagonal graphite such that its edges join seamlessly when the strip is wrapped onto the surface of a cylinder. When x=y, the resultant tube is said to be of the “arm-chair” or (x,x) type, since when the tube is cut perpendicularly to the tube axis, only the sides of the hexagons are exposed and their pattern around the periphery of the tube edge resembles the arm and seat of an arm chair repeated n times. When y=0, the resultant tube is said to be of the “zig-zag” or (x,0) type, since when the tube is cut perpendicular to the tube axis, the edge is a zig zag pattern. Where x≠y and y≠0, the resulting tube has chirality. The electronic properties of the nanotube are dependent on the conformation, for example, arm-chair tubes are metallic and have extremely high electrical conductivity. Other tube types are metallic, semi-metals, or semi-conductors, depending on their conformation. Regardless of tube type, all SWNTs have extremely high thermal conductivity and tensile strength. The SWNT may be a cylinder with two open ends, a cylinder with one closed end, or a cylinder with two closed ends. Generally, an end of an SWNT can be closed by a hemifullerene, e.g. a (10,10) carbon nanotube can be closed by a 30-carbon hemifullerene. If the SWNT has one or two open ends, the open ends can have any valences unfilled by carbon-carbon bonds within the single wall carbon nanotube filled by bonds with hydrogen, hydroxyl groups, carboxyl groups, or other groups. SWNTs can also be cut into ultra-short pieces, thereby forming US-tubes. As used herein, the term “US-tubes” refers to ultra short carbon nanotubes with lengths from about 20 nm to about 100 nm.

The Cₙ can be substituted or unsubstituted. By “substituted” it is meant that a group of one or more atoms is covalently linked to one or more atoms of the Cₙ. Generally, in situ Bingel chemistry may be used to substitute the Cₙ with appropriate groups to form the targeted nanostructures of the present disclosure. Examples of groups suitable for use include, but are not limited to, malonate groups, serinol malonates, groups derived from malonates, serinol groups, carboxylic acid, polyethyleneglycol (PEG), and the like. In one embodiment, the Cₙ is substituted with one or more water-solubilizing groups. Water-solubilizing groups are polar groups (that is, groups having a net dipole moment) that render the generally hydrophobic fullerene core soluble in water. The addition of such groups allow for greater biocompatibility of the Cₙ. Generally, the Cₙ may contain from 1 to 4 addends. The Cₙ can be substituted with any water solubilizing group to allow for sufficient water solubility and biocompatibility, but the spectroscopic properties of the Cₙ should not be compromised. In certain embodiments, the Cₙ may be further substituted with either a thiol (—SH) or an amine (—NH₂) group to aid in the coupling of the crosslinker to the Cₙ moiety.

The crosslinker may comprise any group capable of linking the Cₙ to a drug or to a targeting agent or both. The crosslinker may be covalently bound to the drug or the crosslinker may be covalently bound to the portion of the targeting agent containing the antigen bonding site and capable of associating with the Cₙ, or both. The crosslinker may be physically associated with the Cₙ. Examples of crosslinkers suitable for use in conjunction with the compositions of the present disclosure include but are not limited to, succinate, N-succinimidyl-3-(2-pyridyldithio)-propionate (SPDP), and serinol. In certain embodiments, the crosslinker may be attached directly to an amine substituted Cₙ moiety; in other embodiments, the crosslinker may be used to derivatize the drug and may be attached to an amine substituted Cₙ moiety. In certain embodiments, the crosslinker may be attached directly to an amine substituted Cₙ moiety; in other embodiments, the crosslinker may be used to derivatize the targeting agent and attached to a thiol substituted Cₙ moiety.

The drug used in conjunction with the conjugates of the present disclosure may be attached to the fullerene molecule by the crosslinker. The drug may be used to treat any disease in humans or in animals. An example of one such disease is cancer. As used herein, the term “cancer” refers to an abnormal growth of cells which tend to proliferate in an uncontrolled way, including neoplasms, tumors, and leukemia. In certain embodiments, the drug may be used for the treatment of lung cancer. An example of drugs suitable for use in the compositions of the present disclosure include, but are not limited to, paclitaxel, 20-S-camptothecin, 9-nitro-camptothecin, 9-amino-camptothecin, 10, 11-methylenedioxy-camptothecin, taxol, taxol-A, mitotane, methotrexate, mercaptopurine, lomustine, interferon, 5-fluorouracil, etopiside, cis-platin, carboplatin, and oxaliplatin. In certain embodiments, combinations of drugs may be attached to a single Cₙ molecule. For example, a combination of taxol, 5-fluorouracil, and cis-platin may be used.

As mentioned above, the targeting agent used in conjunction with the present disclosure may be attached to a Cₙ by a crosslinker. The targeting agent may be a protein, an antibody, or a portion of an antibody, such as a glycogen IIa/IIB receptor antibody, Von Willebrand's factor antibody, an antitumor antibody, hepatic cellular antibody, a white blood cell antibody, and antifibrin. Examples of moieties comprising antigen-binding sites that may be used as targeting agents include, but are not limited to, monoclonal antibodies, polyclonal antibodies, Fab fragments of monoclonal antibodies, Fab fragments of polyclonal antibodies, Fab2 fragments of monoclonal antibodies, and Fab2 fragments of polyclonal antibodies, among others. Single chain or multiple chain antigen-recognition sites can be used. Multiple chain antigen-recognition sites can be fused, joined by a linker, or unfused and unlinked.

The targeting agent can be selected from any known class of antibodies. Known classes of antibodies include, but are not necessarily limited to, IgG, IgM, IgA, IgD, and IgE. The various classes also can have subclasses. For example, known subclasses of the IgG class include, but are not necessarily limited to, IgG1, IgG2, IgG3, and IgG4. Other classes have subclasses that are routinely known by one of ordinary skill in the art.

Similarly, the targeting agent can be derived from any species. “Derived from,” in this context, can mean either prepared and extracted in vivo from an individual member of a species, or prepared by known biotechnological techniques from a nucleic acid molecule encoding, in whole or part, an antibody peptide comprising invariant regions which are substantially identical to antibodies prepared in vivo from an individual member of the species or an antibody peptide recognized by antisera specifically raised against antibodies from the species. Exemplary species include, but are not limited to, human, chimpanzee, baboon, other primate, mouse, rat, goat, sheep, and rabbit, among others known in the art. In certain embodiments, the targeting agent may be chimeric, i.e., comprises a plurality of portions, wherein each portion is derived from a different species. A chimeric antibody, wherein one of the portions is derived from human, can be considered a humanized antibody.

Targeting agents are available that recognize antigens associated with a wide variety of cell types, tissues, and organs, and a wide variety of medical conditions, in a wide variety of mammalian species. Examples of medical conditions include, but are not limited to, cancers, such as lung cancer, oral cancer, skin cancer, stomach cancer, colon cancer, nervous system cancer, leukemia, breast cancer, cervical cancer, prostate cancer, and testicular cancer; arthritis; infections, such as bacterial, viral, fungal, or other microbial infections; and disorders of the skin, the eye, the vascular system, or other cell types, tissues, or organs; among others.

Examples of targeting agents include, but are not limited to, those derived from antibodies against anthrax or other bacteria, antibodies against the spores of anthrax or other bacteria, antibodies against vascular endothelial growth factor receptor (VEGF-r) (available from Imclone, New York, N.Y.), antibodies against epidermal growth factor receptor (EGF-r) (available from Abgenix, Fremont, Calif.), antibodies against polypeptides associated with lung cancers (available from Corixa Corporation, Seattle, Wash.), antibodies against human tumor necrosis factor alpha (hTNF-.alpha.) (available from BASF A. G., Ludwigshafen, Germany), among others known in the art.

Suitable targeting agents can be prepared by various techniques that are known in the art. These techniques include, but are not limited to, the immunological technique described by Kohler and Milstein in Nature 256, 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA techniques described by Huse et al. in Science 246, 1275-1281 (1989); among other techniques known to one of ordinary skill in the art.

In addition to the listed antibodies, the targeting agent can be constructed to recognize a target antigen associated with a solid tumor. For example, the targeting agent can be constructed to recognize BER2/neu, MUC-1, HMFG1, or EGFr, associated with breast tumors; MMP-9, BER2/neu, or NCAM, associated with lung tumors; BER2 or 171A, associated with colon tumors; gp240, gangliosides, or integrins, associated with melanomas; BER2 or CA-125, associated with ovarian tumors; or EGFr or tenascin, associated with brain tumors. In certain embodiments, the targeting agent may comprise ZME-018 monoclonal antibody against gp240 in melanoma cells.

Additionally, in certain embodiments, the nanostructure-drug conjugates of the present disclosure may be enclosed within a liposome or a lipid complex. The term “liposome” as used herein, refers to structures having lipid containing membranes enclosing an aqueous interior. An example of one lipid that may be used to form the liposomes of the present disclosure is dilauroylphosphatidylcholine (DLPC). One of ordinary skill in the art, with the benefit of this disclosure, will be able to determine other liposomes suitable for use in conjunction with the compositions and methods of the present disclosure.

Generally, the nanostructure-drug conjugates of the present disclosure may be synthesized using Bingel-Hirsch additions to the carbon nanostructure. In forming liposome encapsulated nanostructure-drug conjugates, the nanostructure-drug conjugates may be dissolved in a solution containing the lipid component of the liposome. The drug-phospholipid mixture may be frozen and lyophilized to form a dry powder. The powder may be resuspended in water to form a suspension of the liposome encapsulated nanostructure-drug conjugates.

In certain embodiments, the nanostructure-drug conjugates of the present disclosure may be used for treating a disease. For example, the nanostructure-drug conjugates of the present disclosure may be used for the treatment of, among other things, lung cancer. In these embodiments, the nanostructure-drug conjugate may be delivered to the respiratory tract of an individual in need of such treatment via small particle aerosol as aqueous dispersions of liposome enclosed nanostructure-drug conjugates. As used herein, the term “aerosol” refers to a suspension of solid or liquid particles in a gas.

It is contemplated specifically that certain compositions of the present disclosure be used for aerosol delivery of aqueous dispersions of liposomes carrying anti-cancer drugs to the respiratory tract. A person having ordinary skill in this art would readily be able to determine, without undue experimentation, the appropriate dosages of these aerosol formulations. When used in vivo for therapy, the aerosol formulations of the present disclosure are administered to the patient in therapeutically effective amounts; i.e., amounts that eliminate or reduce the tumor burden. As with all pharmaceuticals, the dose and dosage regimen will depend upon the nature of the cancer (primary or metastatic), the characteristics of the particular drug (e.g., its therapeutic index), the patient, the patient's history, and other factors. Dose and dosage regimen will vary depending on a number of factors known to those skilled in the art. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press.

In certain embodiments, the present disclosure provides a kit for delivering a nanostructure-drug conjugate through an inhalation route to a mammal which comprises: a) a liposome encapsulated nanostructure-drug conjugate; and, b) a device that forms a liposome aerosol from the composition, for inhalation by the mammal. As used herein, the term “liposome aerosol” refers to aqueous droplets within which are dispersed one or more particles of liposomes or liposomes containing one or more medications intended for delivery to the respiratory tract of a mammal.

Typically, the device contained in the kit comprises: a) an element for heating the liposome encapsulated nanostructure-drug conjugates to form a vapor; b) an element allowing the vapor to cool to form an aerosol; and, c) an element permitting the mammal to inhale the aerosol. In certain embodiments, the mammal may be a human in need of treatment for a disease. In certain embodiments, the disease may be lung cancer.

To facilitate a better understanding of the present invention, the following examples of specific embodiments are given. In no way should the following examples be read to limit or define the entire scope of the invention.

### EXAMPLES

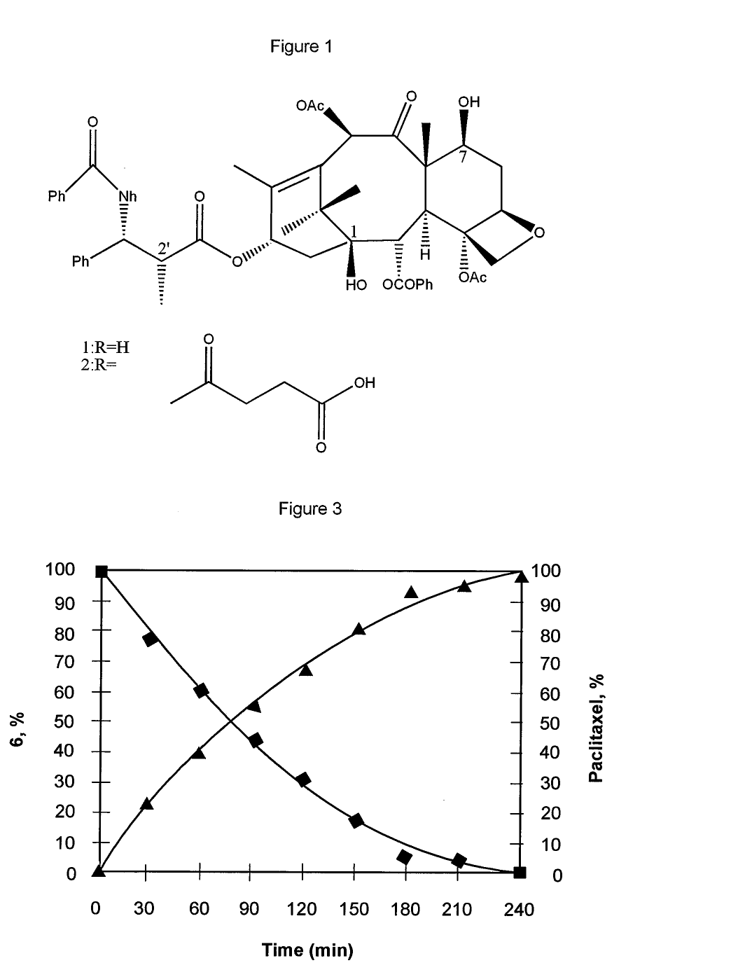

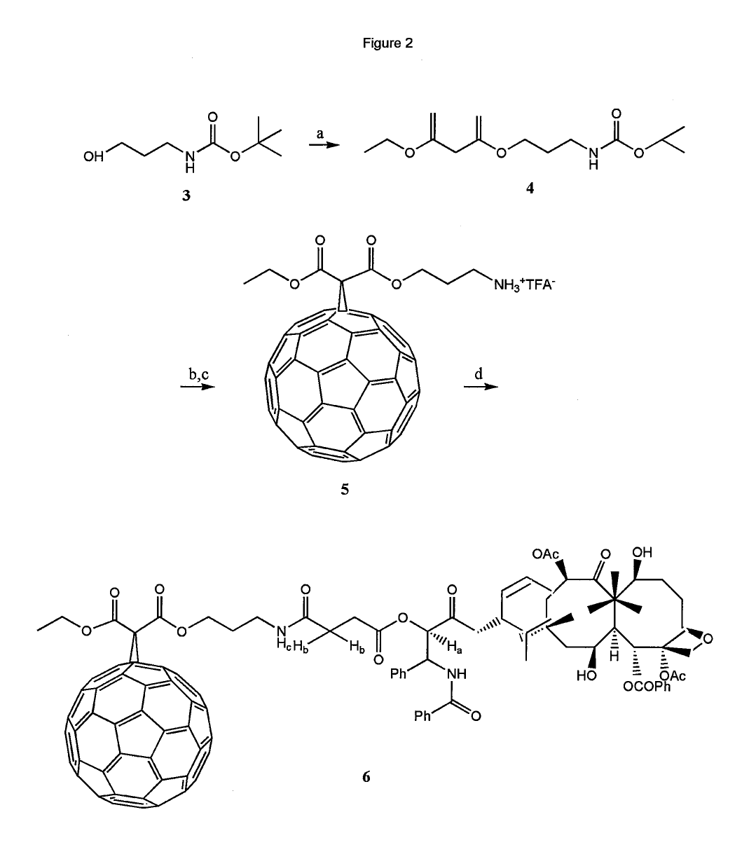

The extensive reports describing structure-activity relationships for paclitaxel were used to suggest a successful approach for the conjugate design. All modifications of the 2′-hydroxyl group of paclitaxel reported so far have resulted in loss of biological activity of the derivatives, except for the ones which contained groups such as esters or carbonates that can be cleaved by enzymatic or other physicochemical mechanisms. Similar potencies and selectivities of the latter prodrugs and paclitaxel itself, as well as isolation of paclitaxel from aqueous solutions of these prodrugs under conditions appropriate for cell culture experiments, are consistent with a mechanism of action dependent on paclitaxel release. Since it has also been established that the 2′-hydroxyl group is more reactive than the 1- and 7-OH groups, we selected this position for modification to an ester with further coupling to a fullerene amino derivative (5, FIG. 2) through a spacer containing a free carboxyl group (2, FIG. 1). Succinate was used as a linker because derivatization of paclitaxel with succinic anhydride has been shown to proceed in high yield.

Synthesis of the C₆₀-paclitaxel conjugate was initiated with the asymmetrical malonate (4) available by treatment of ten-butyl N-(3-hydroxypropyl)carbamate (3) with ethylmalonyl chloride (FIG. 2). Bingel-Hirsh addition to C₆₀, followed by deprotection of the amino group, gave 5. Paclitaxel-2′-succinate (2) was prepared according to the published procedure and coupled to 5 using EEDQ to yield 6.

Spectroscopic and MALDI-TOF MS data for 6 are consistent with the assigned structure. The presence of the conjugate was verified by MALDI-TOF MS with the molecular ion peak at m/z=1844. The ¹HNMR of 6 in CDCl₃ displayed resonances at δ 5.47 ppm (Hₐ in FIG. 2), which confirmed the presence of a 2′-ester group, and at δ 2.79 (H_{b} in FIG. 2) and 6.01 ppm (H_{c} in FIG. 2), which proved amide formation upon EEDQ coupling.

It has been suggested previously that the biodistribution and biological activity of fullerene derivatives depend on their derivatization and aggregation state. Thus, the aggregation properties of 6 and paclitaxel were studied as a function of concentration in aqueous solution (10% DMSO) using a Brookhaven 90Plus submicrometer particle-size analyzer. For 6, the average hydrodynamic diameter (Dₕ) varied from 120 to 145 nm for the concentration range 0.004-0.05 μg/mL and was found to be essentially invariant with concentration. The aggregate sizes were broadly distributed for all concentrations, with a polydispersity index (PDI) between 0.35 and 0.45 nm; the data clearly displayed a bimodal distribution. The intensity of light scattering decreased with decreasing concentration, which can be attributed to a decrease in the concentration of the aggregates without affecting the particle size. These data are in striking contrast to the aggregation behavior of paclitaxel itself, for which 4 and 0.04 μg/mL solutions did not show any scattering. Thus, the C₆₀ component of 6 greatly increases the tendency of paclitaxel to aggregate in aqueous (10% DMSO) solution.

The C₆₀-paclitaxel conjugate (6) is stable in the solid state and in aprotic organic solvents as well as in aqueous media (10% DMSO) at physiological pH. However, incubation of 6 with bovine plasma at 37° C. resulted in the release of paclitaxel, with the half-life of hydrolysis around 80 min (FIG. 3). Assuming a similar half-life for 6 in vivo and the ability of 6 to remain in lungs, a several-fold increase in the exposure time of cancer cells to the drug should be achieved by 6 since the half-life of paclitaxel itself in the lungs has been reported to be only 20 min after delivery by aerosol.

Finally, (6) was examined for its ability to form stable dilauroylphosphatidylcholine (DLPC) liposome formulations and its antitumor activity against human epithelial lung carcinoma A549 cells as a liposome suspension. DLPC is desirable for aerosol delivery of lipophilic agents because it has a low transition temperature (about 0-5° C.), similar to the fluidity of phosphatidylcholine in mammalian cell membranes. To prepare the 6-DLPC liposomes, 1 mg of 6 was dissolved in 10-20 μL of DMSO and mixed with 1 mL of tert-butyl alcohol containing 10 mg of DLPC. The drug-phospholipid mixture was then frozen at −80° C. and lyophilized to a dry powder. Before use, it was resuspended in sterile water and vortexed to form a homogeneous liposomal suspension which was examined by microscopy under polarized light. The suspension was found to be stable with no evidence for drug precipitation. The mean diameter of 6-DLPC liposomes was found to be 2.77 μm, as measured by light scattering (NICOMP). The IC₅₀ value for 6-DLPC was determined through an experiment designed to compare this value with IC₅₀ values for paclitaxel-DLPC, 5-DLPC, and DLPC-only liposomes, prepared as described for 6-DLPC. Cells were exposed to different concentrations of these formulations for 1 h, medium was replaced with drug-free medium, and growth was compared to that of untreated control cells after 2 days of additional incubation. 5-DLPC and DLPC-only did not show any cytotoxicity in the studied concentration range. The mean IC₅₀ values for 6-DLPC and paclitaxel-DLPC were 410 and 253 nM, respectively, revealing similar potencies of these formulations. Thus, it seems reasonable to expect sufficient concentrations of paclitaxel, delivered by 6-DLPC to lungs by small-particle aerosol, to be therapeutic against lung cancer.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

Koshkina, N. V.; Waldrep, J. C.; Knight, V. Curr Cancer Drug Targets 2003, 3, 251-264.
Knight, V.; Koshkina, N. V.; Waldrep, J. C.; Giovanella, B. C.; Gilbert, B. E. Cancer Chemother. Pharmacol. 1999, 44, 177-186.
Koshkina, N. V.; Waldrep, J. C.; Roberts, L. E.; Golunski, E.; Melton, S.; Knight, V. Clin. Cancer Res. 2001, 7, 3258-3262.
Dugan, L. L.; Turetsky, D. M.; Du, C.; Lobner, D.; Wheeler, M.; Almli, C. R.; Shen, C. K.; Luh, T.-Y.; Choi, D. W.; Lin, T.-S. Proc. Natl. Acad. Sci. U.S.A. 1997, 94, 9434-9439.
Friedman, S. H.; DeCamp, D. L.; Sijbesma, R. P.; Srdanov, G.; Wudl, F.; Kenyon, G. L. J. Am. Chem. Soc. 1993, 115, 7918.
Mirakyan, A. L.; Wilson, L. J. J. Chem. Soc., Perkin Tram. 2 2002, 1173-1176.
Wharton, T.; Wilson L. J. Bioorg. Med. Chem. 2002, 10, 3545-3554.
Gautam, A.; Koshkina, N. V. Curr. Cancer Drug Targets 2003, 3, 1-19.
Liebmann, J. E.; Cook, J. A.; Lipschultz, C.; Teague, D.; Fisher, I; Mitchell, J. Br. J. Cancer 1993, 68, 1104-1109.
Kingston, D. G. I. Chem. Comm n. 2001, 867-880. (b) Kingston, D. G. I.; Magri, N. F. J. Nat. Prod. 1988, 51, 298-306. (c) De Groot, F. M. H.; Van Berkom, L. W. A.; Scheeren, H. W. J. Med. Chem. 2000, 43, 3093-3102.
Nicolaou, K. C.; Rlemer, C.; Kerr, M. A.; Rideout, D.; Wrasidlo, W. Nature 1993, 364, 464-466.
Liu, C.; Strobl, J. S.; Bane S.; Schilling J. K.; McCracken M.; Chartterjee S. K.; Rahim-Bata R.; Kingston D. G. I. J. Nat. Prod. 2004, 67, 152-159.
Deutch, H. M.; Glinski, J. A.; Hernandez, M.; Haugwitz, R. D.; Narayanan, V. L.; Suffness, M.; Zalkow, L. H. J. Med. Chem. 1989, 32, 788-792.
Sitharaman, B.; Bolskar, R. D.; Rusakova, I.; Wilson, L. J. Nano Lett. 2004, 4, 2373-2378.
Waldrep, J. C.; Scherer, P. W.; Hess, G. D.; Black, M.; Knight, V. J. Aerosol Med. 1994, 7, 135-145.

What is claimed is:

1. A nanostructure-drug conjugate comprising: a Cₙ, a crosslinker, and a drug, wherein Cₙ refers to a fullerene moiety or nanotube comprising n carbon atoms.

2. The nanostructure-drug conjugate of claim 1 further comprising a targeting agent.

3. The nanostructure-drug conjugate of claim 1 further comprising a targeting agent, wherein the targeting agent comprises an antibody.

4. The nanostructure-drug conjugate of claim 1 wherein the Cₙ is a buckminsterfullerene, single walled carbon nanotube (SWNT), or an ultra-short carbon nanotube.

5. The nanostructure-drug conjugate of claim 1 wherein the Cₙ is substituted with malonate groups, serinol malonates, groups derived from malonates, serinol groups, carboxylic acid, polyethyleneglycol (PEG), amine groups, thiol groups, or combinations thereof.

6. The nanostructure-drug conjugate of claim 1 wherein the crosslinker is N-succinimidyl-3-(2-pyridyldithio)-propionate (SPDP), serinol, succinate, or combinations thereof.

7. The nanostructure-drug conjugate of claim 1 wherein the drug is a drug against lung cancer.

8. The nanostructure-drug conjugate of claim 1 wherein the Cₙ is a buckminsterfullerene.

9. The nanostructure-drug conjugate of claim 1 wherein the drug is paclitaxel.

10. The nanostructure-drug conjugate of claim 1 enclosed within a liposome.

11. A method of treating cancer comprising administering a therapeutically effective amount of a nanostructure-drug conjugate comprising: a Cₙ, a crosslinker, and a drug, wherein Cₙ refers to a fullerene moiety comprising n carbon atoms to a mammal.

12. The method of claim 8 wherein the nanostructure-drug conjugate is administered by aerosol delivery.
